(19) United States
(12) Patent Application Publication
(10) Pub. No.: US 2017/0360367 A1
(43) Pub. Date: Dec. 21, 2017

(54) METHOD FOR MONITORING THE EVOLUTION OF AN INDICATOR USED TO INDICATE THE PHYSICAL STATE OF AN INDIVIDUAL, AND MONITORING DEVICE

(71) Applicant: LSEE, Ajaccio (FR)

(72) Inventors: Cyril TORRE, Paris (FR); Matthieu SERGENT, Toulouse (FR)

(21) Appl. No.: 15/534,238

(22) PCT Filed: Dec. 17, 2015
(86) PCT No.: PCT/FR2015/053562

§ 371 Date: Jun. 8, 2017

(30) Foreign Application Priority Data
Dec. 19, 2014 (FR) 1462862

(51) Int. Cl. A61B 5/00; G01N 33/49; G01N 33/493; G06F 19/00

(52) U.S. Cl. CPC A61B 5/4872; G06F 19/3418; G01N 33/49; G01N 33/493

(57) ABSTRACT

The invention relates to a method for monitoring the evolution of an indicator used to indicate physical state comprising the body fat of a subject and/or the muscular mass of a subject, comprising periodical steps of analysis of a biological fluid for the quantification of a biomarker representative of a metabolic activity comprising lipolysis, myolysis, lipogenesis or myogenesis, and of transmission of the result of said analysis to a remote connected apparatus.

### FIELD OF THE INVENTION

The present invention relates to the field of methods for monitoring the metabolism of a subject and associated connected devices. The invention also relates to devices enabling people to be attentive to their diet.

### BACKGROUND OF THE INVENTION

Obesity has become a major public health issue. It can be defined as an abnormal or excessive accumulation of fat that represents a risk to health and affects the quality of life. Obesity is a major risk factor for diabetes, cardiovascular diseases or cancer. In 2014, 1.4 billion adults were overweight, which caused 3.4 million deaths worldwide each year. Once reserved for the rich countries, it also grows drastically in the emerging countries.

Obesity mainly results from imbalance between an excessive intake of food and insufficient energy expenditure.

Food is a fundamental vector of the individuals' health status. Now its importance in medical practices is under-estimated or misunderstood. Many solutions have been developed to fight obesity, such as diets, coaching, sports, slimming pills, surgery, appetite suppressants . . . . Now, the use thereof often fails because of their unsuitability for specific individuals.

In recent years, the concept of personalized medicine has emerged. Such medicine aims at treating individuals by integrating all their biological data and their individual physiological characteristics. Personalized medicine has naturally extended to nutrition, and focused on food again. Personalized diets based on the individuals' metabolism were historically reserved for top athletes in order to optimize their physical performances. This involved a very burdensome tracing of metabolic changes, specifically using invasive tests ranging from blood tests to biopsy, and not excluding microdialysis.

The present invention proposes to enable as many people as possible to monitor their metabolism in order to guide their decisions as regards food and physical activities according to the goals they have set their minds on.

### PRIOR ART

Connected objects are known from the prior art which make it possible to monitor, for instance an individual's weight or the efforts such individual makes. Such objects include connected scales or bracelets which enable the user to trace one or more parameters such as weight, energy expenditure, sleep, heart rate . . . .

Patent application US2003/0223905 relates to a weight loss system and a method for measuring one or more metabolic parameter(s) in a body fluid sample and correlating the level of the metabolic parameter to a change in body fat or a metabolic state. This system makes it possible to qualify the subject's energy balance, with such energy balance being the amount of energy ingested relative to the amount of energy expended by tracking a marker of lipolysis such as a ketone body, glycerol or a free fatty acid.

A metabolic energy monitoring system having platforms for measuring physiological parameters for calculating energy balance on the basis of kilocalorie intake and energy expenditure is also known from the prior art in the patent application WO2009/131664. This device is preferably continuously worn on the body for a continuous measurement.

### Drawbacks of the Prior Art

Prior art solutions have the drawback of focusing on weight loss and only reflect the body fat mass compared to the calculated energy balance.

Now the metabolic activity is a combination of various parameters, the monitoring of which cannot be limited only to body fat mass and the evolution thereof.

Moreover, the devices of the prior art focus on the quantification of energy as calories burned and ingested in order to give the user recommendations, with such quantification being based on algorithms that do not take the metabolic characteristics specific to each individual into account.

Now, these recommendations are not necessarily suitable because they are based on general concepts which do not directly result from the study of metabolic needs and the individual metabolic characteristics.

### Solution Provided by the Invention

In order to overcome these drawbacks, the present invention in its broadest sense, provides a method for monitoring the evolution of an indicator used to indicate a physical state comprising the body fat of a subject and/or the muscular mass of a subject, comprising periodic steps of analysis of a biological fluid for the quantification of a biomarker representative of a metabolic activity comprising lipolysis, myolysis, lipogenesis or myogenesis and of transmission of the result of said analysis to a remote connected apparatus.

It should be understood that the present invention makes it possible to monitor the metabolism of a subject over a given period of time at a frequency freely selected by the user. The user can at first use the invention to understand his/her metabolism and correlate his/her eating and behavioral habits and his/her physical activity at a measured level of metabolism, for example lipolysis or myolysis. The user can capitalize the data in order to establish control references and so be able to adapt his/her diet or practice adapted physical activity based on the chosen goal. The present invention thus has the advantage that the control values are directly related to the individual by the constitution of such references, and not to repositories or methods for calculating the energy expended according to generally accepted methods.

It should be understood that the method according to the invention comprises:

periodic steps of analyzing a biological fluid for the quantification of a biomarker representative of a metabolic activity comprising lipolysis, myolysis, lipogenesis and myogenesis, with said analysis being performed before, during and/or after given physical activity, eating habit and/or behavior

transmitting the result of said analysis to a remote connected apparatus

establishing the subject's individual repository correlating physical activity, eating habits and/or behavior with the quantification of said biomarker

comparing the quantification of a biomarker with the individual repository.

The method may further comprise a step of identifying, among such individual repository, the physical activity, eating habits and/or the most appropriate behavior in relation to the goal that the subject has set for himself/herself. This objective can be the muscular mass to be reached, a body weight to be reached, a body fat to be reduced, a metabolic activity to be maintained . . . .

It should be understood that after making up his/her own individual repository, the subject can compare his/her subsequent performances and monitor his/her metabolic activity.

Thus, the invention belongs to customized medicine which places food at the center of individuals' health and well-being and easily studies objectively measurable and quantifiable parameters, which can be correlated with the intensity of metabolism, particularly lipolysis, myolysis and their counterparts, i.e. lipogenesis and myogenesis.

Biomarker should be understood as molecules circulating and/or ending up in a biological fluid selected from blood, urine, sweat, saliva or tears. Advantageously, the biological fluid is urine or blood according to the various embodiments.

Such biomarkers can be by-products of the degradation of muscles and/or body fat. The concentration of such biomarkers varies depending on the subject's metabolism, reflecting the lipids metabolism (lipolysis/lipogenesis) or the muscle metabolism (myolysis/myogenesis).

According to one embodiment, the biomarker detected in urine is selected from glycerol, carnitine, acyl-carnitine, a ketone body, beta aminoisobutyrate, a marker of inflammation and the metabolites thereof.

According to another embodiment, the biomarker detected in blood is selected from free fatty acids, glycerol, carnitine, acyl-carnitine, a ketone body, beta aminoisobutyrate, a marker of inflammation and the metabolites thereof.

According to a particular embodiment, the marker of inflammation detected in the biological fluid is an interleukin, preferably interleukin 6.

The invention also relates to an apparatus for implementing the method according to the invention. The apparatus comprises means for analyzing a biological fluid for the quantification of a biomarker, a local clock and a calculator running a computer program for controlling said analyzing means, processing the information provided by the analyzing means, recording in a local memory, and communication with a connected device.

The means for analyzing a biological fluid comprises means for receiving the biological fluid such as a reservoir or an absorbent material. Such receiving means may be coupled to biosensors to quantify the biomarker, such as antibodies or enzymes.

Such receiving means, whether coupled or not to biosensors, may be disposable or cleanable. They are preferably disposable.

The device includes means for communicating data to a remote connected apparatus, preferably wireless communicating means such as Bluetooth, WiFi, internet or infrared beam.

According to another aspect, the invention provides for a system for monitoring an indicator used to indicate a physical state comprising a subject's body fat and/or muscular mass, with the system comprising at least one apparatus as defined above and at least one connected apparatus, including a tablet or a cell phone and a computer program controlling the operation of said apparatus connected for communicating with said apparatus, local and/or remote recording the results of the periodic analyses, visualizing the graphic information calculated on the basis of a plurality of recorded analyses.

According to another aspect, the invention relates to a computer program for controlling a device connected to a device as defined above, with the computer program controlling the communication between the connected device and the analyzing device, recording into a memory of the connected device or of a remote server time-stamped and possibly geo-located data and the displaying, on a screen of the connected device, of graphic information calculated on the basis of a plurality of recorded analyses.

### DESCRIPTION

The invention will be better understood when reading the description of non-limiting embodiments.

The device according to the invention makes it possible to measure one or more biomarker(s) in blood or urine. In the examples below, the device can measure, in blood, either glycerol or beta-aminoisobutyrate, or interleukin 6, or a combination thereof.

### Measurement of Glycerol in Blood:

In this exemplary embodiment, the device makes it possible to measure the concentration of glycerol in blood.

The user measures a concentration of glycerol of 0.3 mmol/L at rest, and a concentration of 1 mmol/L after one hour of cycling at moderate speed.

The user repeats this measurement before and after various physical activities. These physical activities can be, for example walking, jogging, swimming or any other physical activity. The user thus makes his/her own repository.

He/she compares the values obtained after various physical activities and identifies the one most suitable for his/her metabolism in order to stimulate the degradation of his/her body fat and/or stimulate the synthesis of his/her muscular mass.

Measurement of beta-aminoisobutyrate in Blood:

The user measures a concentration of beta aminoisobutyrate of 1.8 μmol/L and a concentration of beta aminoisobutyrate of 2.7 mmol/L after an hour of cycling at a moderate speed.

The user repeats this measurement before and after various physical activities. These physical activities can be, for example walking, jogging, swimming or any other physical activity. The user thus makes his/her own repository.

He/she compares the values obtained after various physical activities and identifies the one most suitable for his/her metabolism in order to stimulate the degradation of his/her body fat and/or stimulate the synthesis of his/her muscular mass.

### Measuring a Marker of Inflammation, Interleukin 6 in Blood:

The user measures a concentration of interleukin 6 of 10 ng/L at rest and a concentration of interleukin 6 of 120 ng/L after an hour of cycling at a moderate speed.

The user repeats this measurement before and after various physical activities. These physical activities can be, for example walking, jogging, swimming or any other physical activity. The user thus makes his/her own repository.

He/she compares the values obtained after various physical activities and identifies the one most suitable for his/her metabolism in order to stimulate the degradation of his/her body fat and/or stimulate the synthesis of his/her muscular mass.

### Compilation of Measured Data:

The measurement data of the biomarkers are recorded.

Regarding the indicators listed on the application, the measured raw parameters (including the value at rest) are used to establish an intensity scale representative of a metabolic activity (lipolysis, myolysis . . . ). An arbitrary unit is assigned and a color scale representative of intensity (from red for a very low lipolysis, to green for high intensity) is generated.

The device includes an electronic biosensor for quantifying the biomarker of interest in the biological fluid. Prior to making some physical effort, the user places a sample of a biological fluid on a polymer strip whereon the biosensors are set. Then the user places the strip into the biosensor. The biosensor measures the concentration of the biomarker in the biological sample and records same. The user can then choose to send the measure to a device such as a computer, a mobile phone, a tablet via Bluetooth.

The strip is disposable. It comprises appropriate biosensors according to the biomarker to be quantified.

The measure received by the user's phone, for instance, is integrated into an application which will process same. The user can then view his/her physical condition at the time of measurement. After making a physical effort, the user repeats the same operation and compares the results.

The user thus makes up his/her own database in order to be able to correlate his/her physical state with the food he/she has eaten and his/her physical activity. This data base is his/her own repository. Thanks to the invention, the user knows the impact of a particular food habit or of a particular intensity of physical activity on his/her metabolism. He/she thus has visibility on the practices to be implemented to reach his/her goal, such as a given weight or a particular muscular mass. The user can also monitor the evolution of his/her progress to reach his/her goal.

The user can also combine in this application other parameters from other connected devices, such as a connected scale, for instance, in order to follow the curve of evolution of his/her weight, and also of his/her body fat.

The application also offers recommendations based on the user's repository on the basis of the goal he/she has set for himself/herself.

The user can trace real-time changes in his/her physical state according to the goal he/she has set for himself/herself and especially adapt his/her diet and physical activity on the basis of this goal. The invention thus strongly motivates the user since he/she concretely visualizes in real time his/her progress relative to his/her goal.

### DESCRIPTION OF A NON-LIMITING EXAMPLE OF AN APPARATUS ACCORDING TO THE INVENTION

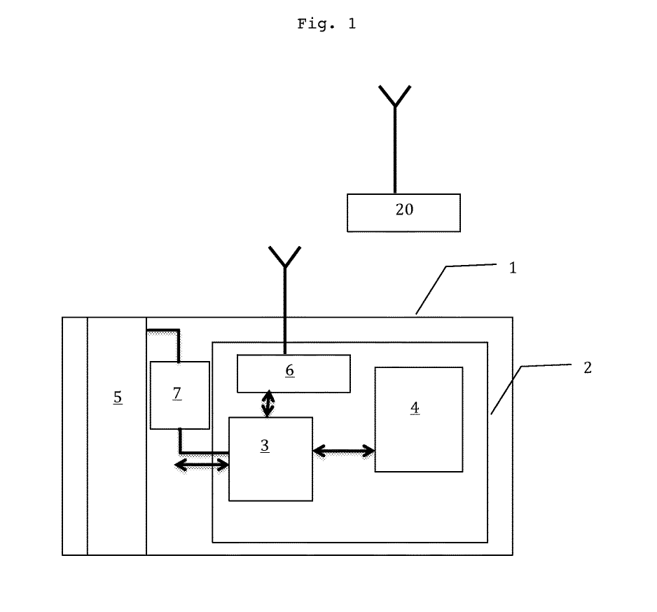

FIG. 1 shows the principle diagram of an apparatus 1 according to a particular embodiment of the present invention. It comprises a micro-computer 2 capable of executing computer instructions and data processing. Such micro-computer 2 comprises a microprocessor 3 which can be of any known type of the state of the art. The micro-computer 2 also comprises a storage unit 4 adapted to receive a computer program defining a set of instructions specific to the implementing of the method, and to store data. A battery 7 supplies power to electrical and electronic circuits of the apparatus.

The apparatus 1 further comprises a detector 5 comprising a strip reader of a known type.

The apparatus 1 also comprises an input-output interface 6 connected to the micro-computer 120 which enables communication with an associated apparatus 20, for example a tablet or a cell phone.

The input-output interface 6 can be of the wired type enabling the connection to the associated apparatus 20 via a cord provided with a connector suitable for the associated apparatus 20. However, in order to avoid connection compatibility issues, the input-output interface 6 is rather of the radiofrequency type, e.g. of the Bluetooth type.

For Bluetooth communication, establishing a connection starts with a phase of inquiry, during which the computer program stored in the memory 4 controls the sending of a query to all the apparatuses present in the coverage area, so-called access points. All the peripheral devices receiving the query respond with their addresses.

The apparatus 1 selects an address previously recorded or selected by the user and synchronizes with the access point using a so-called paging technique, which consists in synchronizing its clock and frequency with the access point.

A link is then established with the access point, which enables the apparatus 1 to initiate a phase of discovery of the services of the access point, using a so-called SDP (Service Discovery Protocol).

Upon completion of this phase of service discovery, the master device is able to create a communication channel with the access point using the L2CAP protocol.

In order to protect sensitive data, including historical data recorded in the memory 4 during previous acquisitions, an additional RFCOMM channel, operating above the L2CAP channel is established to provide a virtual serial port.

The associated apparatus 20 preferably includes a pairing security mechanism, making it possible to restrict access to authorized users only in order to guarantee some tightness to the piconet. Pairing is done using an encryption key commonly known as “PIN” (Personal Information Number). The associated apparatus 20 thus sends a request for pairing to the master device. This can, most of the time, prompt an intervention by the user to enter the PIN code of the access point. If the received PIN code is correct, the association is obtained.

In secure mode, the PIN will be sent encrypted using a second key, in order to avoid any risk of compromise.

When pairing is effective, the associated apparatus 20 is free to use the communication channel thereby established in order to recover the data recorded in the memory 4 of the device 1 and ensure a communication with a remote server through a digital telecommunications network (e.g. Internet 2 0) preferably in a secured manner. Such remote server records the data provided by the device 1 by associating same with time-stamping and/or geo-location data from the associated apparatus 20 so as to form a historical data base for the data relative to the user identified by the device 1 and process same to provide data representative of the evolution of an indicator of the user's physical state.

Optionally, after said processing, the server returns the result of the analysis to the user via the same network or another one. Optionally, the server records the data and/or the analysis result on a recording medium. Of course, means for guaranteeing the anonymity of the donor's and the recipient's physiological/clinical characteristics can be provided for.

The device 1 enables the recording of a user's identifier, or a plurality of identifiers each corresponding to a given user.

This identifier is used by the associated apparatus 20 to connect with the server and to enable the configuration of the user's personal data within an application loaded in the memory of the associated apparatus 20 and on the remote server.

1-9. (canceled)

10. A method for monitoring the evolution of an indicator used to indicate a physical state comprising the body fat of a subject and/or the muscular mass of a subject, comprising periodical steps of analysis of a biological fluid for the quantification of a biomarker representative of a metabolic activity comprising lipolysis, myolysis, lipogenesis or myogenesis, and of transmission of the result of said analysis to a remote connected apparatus.

11. A monitoring method according to claim 10, characterized in that said biological fluid is selected from blood, urine, sweat, saliva or tears.

12. A monitoring method according to claim 10, characterized in that said biological fluid is urine.

13. A monitoring method according to claim 10, characterized in that said biological fluid is blood.

14. A monitoring method according to claim 10, characterized in that the biomarker detected in urine is selected from glycerol, carnitine, acyl-carnitine, a ketone body, beta aminoisobutyrate, a marker of inflammation and the metabolites thereof.

15. A monitoring method according to claim 10, characterized in that the biomarker detected in blood is selected from free fatty acids, glycerol, carnitine, acyl-carnitine, a ketone body, beta aminoisobutyrate, a marker of inflammation and the metabolites thereof.

16. A device for implementing the method according to claim 10, characterized in that it comprises means for analyzing a biological fluid for the quantification of a biomarker, a local clock and a calculator running a computer program for controlling said analyzing means, processing the information provided by the analyzing means, recording in a local memory, and communication with a connected device.

17. A system for monitoring an indicator used to indicate the physical state comprising the body fat of a subject and/or the muscular mass of a subject, characterized in that it comprises at least one device according to claim 16 and least one connected device, specifically a tablet or a cell phone, and a computer application controlling the operation of said connected device for communicating with said device, local and/or remote recording of the results of the periodic analyses, visualizing the graphic information calculated on the basis of a plurality of recorded analyses.

18. A computer program product for controlling a device connected to a device according to claim 16, characterized in that it controls the communication between the connected device and the analysis equipment, the recording into a memory of the connected device or of a remote server of time-stamped and possibly geo-located data and the displaying, on a screen of the connected device, of graphic information calculated on the basis of a plurality of recorded analyses.
